(19) United States
(12) Patent Application Publication
(10) Pub. No.: US 2011/0243938 A1
(43) Pub. Date: Oct. 6, 2011

(54) Methods to Reduce B-Helper T Cells to Treat Autoimmune Diseases

(75) Inventors: Hideki Ueno, Dallas, TX (US); Jacques F. Banchereau, Dallas, TX (US); Maria Virginia Pascual, Dallas, TX (US); Nathalie Schmitt, Dallas, TX (US)

(73) Assignee: Baylor Research Intitute, Dallas, TX (US)

(21) Appl. No.: 13/121,680

(22) PCT Filed: Sep. 29, 2009
(86) PCT No.: PCT/US2009/058891

§ 371 Date: Jun. 10, 2011

Related U.S. Application Data
(60) Provisional application No. 61/101,606, filed on Sep. 30, 2008.

(51) Int. Cl. A61K 39/395; C12N 5/0783; A61K 31/713; A61K 31/7088; A61P 3/10; A61P 19/02; A61P 7/06; A61P 25/00; A61P 29/00; A61P 37/06

(52) U.S. Cl. 424/133.1; 435/377; 424/158.1; 424/145.1; 424/184.1

(57) ABSTRACT

The present invention includes compositions and methods for the treatment of autoimmune diseases by administering to a subject having an autoimmune disorder an effective amount of a therapeutic composition comprising a pharmaceutically acceptable carrier and at least one IL-12 inhibitor, e.g., a blocking anti-IL-12 antibody or fragment thereof.

### TECHNICAL FIELD OF THE INVENTION

The present invention relates in general to the field of modulating immune responses, and more particularly, to compositions and methods for the diagnosis, prevention and treatment of autoimmune diseases by reducing the activity of B-Helper T cells.

### BACKGROUND OF THE INVENTION

Without limiting the scope of the invention, its background is described in connection with treatments against autoimmune responses.

A number of chemical and biological immunotherapeutic methods have been developed for treating autoimmune disorders. Typically, immunotherapeutic methods have attempted to treat autoimmune disorders after they have begun by dampening the entire immune response using, e.g., chemical agents such as steroids or anti-immune cell antibodies. For example, one such method attempts to treat autoimmune disorders by administering antibodies that bind to B-cell antigens, e.g., CD22, CD20, CD19, and CD74 or HLA-DR antigen. However, the antibodies target all B cells without regard to their normal functioning and damped the entire immune response, not just the target autoimmune response.

On such is U.S. Pat. No. 7,074,403, issued to Goldenberg, et al., for immunotherapy of autoimmune disorders using antibodies that target B-cells. This patent teaches the use of antibodies that bind a B-cell antigen to provide an effective means to treat autoimmune disorders. Antibodies and fragments, which may be conjugated or naked, are used alone or in multimodal therapies. The antibodies may be bispecific antibodies and may be produced recombinantly as fusion proteins, or as hybrid, polyspecific antibodies.

U.S. Pat. No. 6,103,713, issued to Ways, et al., teaches a therapeutic treatment for autoimmune diseases by inhibiting activation and/or proliferation of T cells and B cells and for treating autoimmune diseases and/or disease manifestations using the isozyme selective PKC inhibitor: (S)-3,4-[N,N′-1,1′-((2″-ethoxy)-3′″(O)-4′″-(N,N-dimethylamino)-butane)-bis-(3,3′-indoly 1)]-1(H)-pyrrole-2,5-dione and its pharmaceutically acceptable salts.

Another example is United States Patent Application No. 20070025990, filed by Dingivan for methods of administering/dosing CD2 antagonists for the prevention and treatment of autoimmune disorders or inflammatory disorders. The application teaches compositions for the prevention or treatment of an autoimmune disorder or an inflammatory disorder in a subject with one or more CD2 antagonists and methods for preventing or treating an autoimmune disorder or an inflammatory disorder in a subject by administering one or more CD2 binding molecules to said subject. The present invention provides doses of CD2 binding molecules and methods of administration that result in improved efficacy, while avoiding or reducing the adverse or unwanted side effects associated with the administration of an agent that induces the depletion of peripheral blood lymphocytes.

Yet another application is United States Patent Application No. 20040022787, filed by Cohen, et al., for methods for treating an autoimmune disease using a soluble CTLA4 molecule and a DMARD or NSAID. Briefly, compositions and methods are taught for treating immune system diseases such as rheumatic disease, by administering to a subject soluble CTLA4 molecules that block endogenous B7 molecules from binding their ligands, alone, or in conjunction with other agents including Disease Modifying Anti-Rheumatic Drugs (DMARDs).

### SUMMARY OF THE INVENTION

The present invention includes compositions and methods for the regulation of immune responses, and more particularly, to the modulation of B-helper T cell activity. It has been found that IL-12 is a key regulator of B cell proliferation, maturation and activation into immunoglobulin secreting cells. It is demonstrated herein that IL-12 regulates a key population of T cells, B helper T cells, by controlling the development and activation of this T cell subset and their secretion of IL-21. Physiologically is was discovered and it is demonstrated herein that in autoimmune disorders that include autoimmune antibodies, such as juvenile dermatomyositis (JDM), systemic arthritis (SYS), and systemic lupus erythematosus (SLE), that there is an increase in the presence of B-helper T cells in the blood of these autoimmune disease patients. It was further demonstrated that IL-12 is a key molecule in the secretion of IL-21 by T cells from patients with autoimmune diseases.

This invention includes compositions and methods to reduce B-helper T cell numbers or activity to treat autoimmune diseases. One such method includes blocking IL-12 activity to inhibit the development of B-helper T cells and their secretion of IL-21. Another method reduces the numbers of B-helper T cells with monoclonal antibodies, including CXCR5. Yet another method includes reducing the secretion of IL-21 from B-helper T cells by blocking ICOS.

In one embodiment, the present invention includes compositions and methods for treating an autoimmune disorder by administering to a subject having an autoimmune disorder, an effective amount of a therapeutic composition comprising an IL-12 inhibitor in an amount sufficient to decrease B-Helper T cell thereby reducing, e.g., auto-immune antibody secretion by B cells. In one aspect, the IL-12 inhibitor comprises at least one blocking anti-IL-12 antibody or fragment thereof. In another aspect, the method further comprises a pharmaceutically acceptable carrier. In one embodiment, the IL-12 inhibitor comprises an IL-12 inhibitory antibody administered in a dosage of from 1 to 1,000 mg per dose. In one aspect, the IL-12 inhibitor comprises an IL-12 inhibitory antibody and the subject receives the antibody in repeated dosages. In yet another aspect, the IL-12 inhibitor comprises an anti-IL-12 antibody selected from the group consisting of subhuman primate antibody, murine monoclonal antibody, chimeric antibody, humanized antibody, and human antibody. In another aspect, the IL-12 inhibitor comprises an RNAi, siRNA or other nucleic acid inhibitor of IL-12.

Non-limiting examples of autoimmune disease that may be treated with the present invention include those in which, e.g., autoimmune antibodies trigger an autoimmune response, e.g., acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis. In yet another aspect of the present invention, the IL-12 inhibitor may be provided with a secondary therapeutic directed against T-cells, B-cells, plasma cells, or macrophages or inflammatory cytokines.

Yet another embodiment of the present invention is a method of enhancing an antigen-specific B cell response against an antigen comprising: isolating naïve CD4+ T cells; and maturing the naïve CD4+ T cells in the presence of antigen-loaded dendritic cells with an effective amount of IL-12 sufficient to develop and activate B-helper T cells, wherein the IL-12 treated B-Helper T cells secrete nanomolar amounts of IL-21. In one aspect, the naïve CD4+ T cells are isolated from peripheral blood mononuclear cells by negative selection. In another aspect, the naïve CD4+ T cells are obtained from peripheral blood mononuclear cells by negative selection using antibodies against CD8 and one or more antibodies against CD11b, CD11c, CD14, CD15, CD16, CD19, CD45RO, CD56 and HLA-DR. In another aspect, the antigen is selected from a virus, a bacteria, a fungi, a cancer or a toxin.

Another embodiment of the present invention includes compositions and methods of modulating autoimmune diseases comprising: identifying a patient suspected of needing therapy for an autoimmune disorder caused by the secretion of autoimmune antibodies by B cells; and treating the patient with an amount of an anti-IL-12 inhibitor sufficient to inhibit CD4+ B helper T cells. In one aspect, the anti-IL-12 inhibitor comprises an anti-IL-12p40 mAb or an anti-IL-12p70 mAb, an anti-IL-12p70 mAb, anti-IL-12 receptors, soluble inactive IL-12 and combinations thereof. In another aspect, the anti-IL-12 inhibitor comprises an IL-12 receptor antagonist. In yet another aspect, the CD4+ B helper T cells are selected by negative selection. In one aspect, the CD4+ B helper T cells are obtained from peripheral blood mononuclear cells by negative selection using antibodies against CD8 and one or more antibodies against CD11b, CD11c, CD14, CD15, CD16, CD19, CD45RO, CD56 and HLA-DR. In another aspect, the CD4+ B helper T cells are activated in the presence of activated dendritic cells. In one aspect, the autoimmune disease is selected from systemic lupus erythematosus, dermatomyositis, juvenile dermatomyositis, arthritis, systemic arthritis and psoriatic arthritis. In one aspect, the IL-12 inhibitor is provided to a subject suspected of susceptibility of an autoimmune disease prior to the development of autoimmune antibodies.

Another embodiment of the present invention is a B cell Helper T cell made by the method comprising: isolating naïve CD4+ T cells; and maturing the naïve CD4 T cells in the presence of activated dendritic cells expressing a target antigen in the presence of IL-12, wherein the matured CD4+ B-helper T cells release nanomolar amounts of IL-21 in response to antigen.

Another embodiment of the present invention includes compositions and methods of regulating

B cell proliferation, maturation and activation into immunoglobulin secreting cells by exposing IL-21 secreting B-Helper T cells to a composition comprising an IL-12 inhibitor. In one aspect, the IL-12 inhibitor reduces the secretion of both IL-21 and IFN-γ by CD4+ B-Helper T cells. In another embodiment, the present invention includes a method of enriching B Helper T cells comprising incubating naïve CD4 T cells in the with an amount of IL-12 sufficient to trigger the release of nanomolar amounts of IL-21.

### BRIEF DESCRIPTION OF THE DRAWINGS

For a more complete understanding of the features and advantages of the present invention, reference is now made to the detailed description of the invention along with the accompanying figures and in which:

FIG. 1 shows that IL-12 induces naïve CD4+ T cells to secrete IL-21.

FIG. 2 shows that naïve CD4⁺ T cells primed with IL-12 induce B cells to produce Immunoglobulins.

FIG. 3 shows that CD4⁺ T cells primed with IL-12 help B cells through IL-21 and ICOS.

FIG. 4 shows activated DCs induce IL-21-producing CD4⁺ T cells through IL-12.

FIG. 5 shows that blocking IL-12 inhibits the development of T cells capable of helping B cells.

FIG. 6 shows that IL-12 controls the secretion of IL-21 secretion by memory CD4⁺ T cells.

FIG. 7 shows that CD4⁺ T cells and B cells predominantly accumulate at inflammatory sites in DM.

FIG. 8 shows the increased frequencies of functional B helper T cells in the blood of autoimmune disease patients.

FIG. 9 shows the increased IL-21 secretion in response to SEB by peripheral blood mononuclear cells obtained from active JDM patients.

FIG. 10 shows that IL-21 secretion by PBMCs from JDM patients is dependent on IL-12.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In certain embodiments, the present invention the step of identifying that a subject is in need of treatment for a B-cell regulated autoimmune disorder. The identification can be in the judgment of a subject or a health professional and can be subjective (e.g., opinion) or objective (e.g., measurable by a test or a diagnostic method).

As used herein, the terms “treat”, “treatment” and “treating” refer to the reduction or amelioration of the progression, severity and/or duration of a B-cell regulated autoimmune disorder or the amelioration of one or more symptoms (preferably, one or more discernible symptoms) of a B-cell regulated autoimmune disorder resulting from the administration of one or more therapies of the present invention that target autoimmune responses mediated by B-Helper T cells that secrete large amounts of IL-21 in response to activation of the T cells by antigen-loaded antigen presenting cells (e.g., dendritic cells) in the presence of IL-12.

As used herein, the terms “prevent”, “prevention” and “preventing” refer to the reduction in the risk of acquiring or developing a given a B-cell regulated autoimmune disorder, or the reduction or inhibition of the recurrence, onset or development of one or more symptoms of a given a B-cell regulated autoimmune disorder. In one embodiment, an IL-12 inhibitor is administered as a preventative measure to a patient (e.g., a human), suspected of having a genetic predisposition to any of the disorders described herein.

As used herein, the term “effective amount” refers to an amount of an IL-12 inhibitor that is sufficient to reduce or ameliorate the severity, duration, progression, or onset of a B-cell regulated autoimmune disorder, prevent the advancement of an a B-cell regulated autoimmune disorder, cause the regression of a B-cell regulated autoimmune disorder, prevent the recurrence, development, onset or progression of a symptom associated with a B-cell regulated autoimmune disorder, or enhance or improve the prophylactic or therapeutic effect(s) of another therapy. In one embodiment, a treatment according to the invention provides a reduction in, or prevention of, at least one symptom or manifestation of a B-cell regulated autoimmune disorder, as determined in vivo or in vitro of at least about 10%, or even 20%, 30%, 40%, 50%, 60%, 70%, 80%, 90%, 95%, 98% or 99%.

The present invention recognizes the interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 1000 mg/kg, more preferably 0.01 mg/kg to about 100 mg/kg, more preferably 0.1 mg/kg to about 10 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

In operation, the compositions and methods of the present invention may include an anti-IL-12 inhibitor that alone, or as a component of a pharmaceutical composition, can be administered intravenously, orally, parenterally, by inhalation spray, topically, rectally, nasally, buccally, vaginally or via an implanted reservoir. Other non-limiting examples delivery of the IL-12 inhibitors and methods of the present invention include subcutaneous, intracutaneous, intravenous, intramuscular, intraarticular, intraarterial, intrasynovial, intrasternal, intrathecal, intralesional and intracranial injection or infusion techniques. In certain embodiments, the B-Helper T cells may be isolated, treated in vitro, and then returned to the subject for treatment.

For example, the present invention may be prepared and administered as a sterile injectable composition, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

Another example of the present invention is a composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. A compound of this invention can also be administered in the form of suppositories for rectal administration.

As used herein, the term “pharmaceutically acceptable carrier” refers to those agents (active or inactive) in which the IL-12 inhibitor may be incorporated that is not generally deleterious to the subject to be treated. For example, solubilizing agents such as cyclodextrins, that form specific, more soluble complexes with the compounds of this invention, or one or more solubilizing agents, can be utilized as pharmaceutical excipients for delivery of the compounds of the invention. Examples of other carriers include colloidal silicon dioxide, magnesium stearate, cellulose, sodium lauryl sulfate, and dyes.

As used herein, the terms “animal”, “subject,” “mammal” and “patient”, include, but are not limited to, a human or an animal such as a cow, monkey, horse, sheep, pig, chicken, turkey, quail, cat, dog, mouse, rat, rabbit and hamster.

The methods for treating or preventing a B-cell regulated autoimmune disorder in a patient in need thereof can further comprise administering to the patient being administered an IL-12 inhibitor in an effective amount. The IL-12 inhibitor may also be delivered with other therapeutic agents such as those conventionally used to prevent or treat a B-cell regulated autoimmune disorder or symptoms thereof. In such combination therapy treatment, both the IL-12 inhibitor and the other drug agent(s) may be administered to mammals (e.g., humans, male or female) by conventional methods. The agents may be administered in a single dosage form or in separate dosage forms. Effective amounts of the other therapeutic agents are well known to those skilled in the art. In light of the present disclosure, the skilled artisan can determine the other therapeutic agent's optimal effective-amount range. In certain embodiments of the invention, the effective amount of the IL-12 inhibitor of this invention may be reduced where a second therapeutic agent potentiates or enhances the effect of the IL-12 inhibitor.

Examples of agents for combination therapy may include a TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluororquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine or a cytokine antagonistm. Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), relevant portions incorporated herein by reference.

Non-limiting example of autoimmune diseases that may be diagnosed, prevented or treated using the present invention include those autoimmune diseases that may include at least in part or even predominantly immunoglobulin responses against self-antigens, for example, autoimmune disease such as systemic lupus erythematosus, Sjogren's syndrome, rheumatoid arthritis, juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyl), and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

The present inventors have discovered that IL-12 is the key molecule that regulates antibody responses in the human. It was also found that the frequency of functional B-helper T cells is increased in autoimmune diseases, including JDM, SYS, and SLE. Targeting B-helper T cells has never been claimed as a therapeutic approach in autoimmune diseases. Using the present invention, it is now possible to: 1) block IL-12 to inhibit the development of B-helper T cells, 2) block IL-12 to inhibit the secretion of IL-21 by B-helper T cells, and 3) target B-helper T cells to reduce their number in the body, for example by using monoclonal antibodies against CXCR5 or ICOS.

For example, it was found that peripheral blood mononuclear cells (PBMCs) obtained from autoimmune disease patients including dermatomyositis, and systemic lupus erythematosus were cultured with superantigen enterotoxin B (SEB) in the presence or absence of IL-12-netralizing antibody (anti-IL-12p70 mAb), and IL-21 secretion was analyzed at day 2 of cultures. Addition of IL-12-blocking antibody significantly inhibited IL-21 secretion from SEB-reactive CD4+ T cells. Therefore, blocking IL-12 decrease the secretion of IL-21 from CD4+ T cells, which play a major role in the development of antibody responses.

Isolation of naïve CD4⁺ T cells. PBMCs were purified by Ficoll gradient centrifugation from apheresis blood samples obtained from adult healthy volunteers and kept frozen in 10% DMSO in liquid nitrogen. Naïve CD4⁺ T cells were first enriched by negative selection: PBMCs were incubated with purified CD8 (HIT8a, eBiosciences), CD11b (LM1/2, ATCC), CD11c (B-1y6, BD Biosciences), CD14 (M5E2, ATCC), CD15 (W6D3, BD Biosciences), CD16 (3G8, Beckman Coulter), CD19 (J4.119, Beckman Coulter), CD45RO (UCHL1, BD Biosciences), CD56 (C218, Beckman Coulter) and HLA-DR (B8.12.2, Beckman Coulter) mAbs at 4° C. for 30 min, and then incubated with Dynabeads Pan Mouse IgG (Dynal) at 4° C. for 30 min. Antibody-bound cells were removed with magnet (Dynal). Naive CD4⁺ T cells were further purified by sorting with FACSAria (BD Biosciences) as CD8⁻ CD56⁻HLA-DR⁻ CD45RA⁺ CD4⁺ cells after staining with CD8 PE (RPA-T8, eBiosciences), CD56 PE (B159, BD Biosciences), HLA-DR PE (G46-6, BD Biosciences), CD45RA Tricolor (MEM-56, Caltag) and CD4⁺ Pacific Blue (S3.5, Caltag) mAbs. Cell purity was >99%.

Isolation of B cells. B cells were first enriched from apheresis PBMCs by positive selection using CD19 MicroBeads and LS column (Miltenyi Biotec). Then, naïve and memory B cells were sorted with FACSAria as IgD⁺ CD27⁻ CD3⁻ CD11c⁻ CD14⁻ cells and IgD⁻ CD27⁺ CD3⁻ CD11c⁻ CD14⁻ cells, respectively, after staining with IgD FITC (IA6-2, BD), CD27 PE (L128, BD Biosciences), CD3 APC (SK7, BD Biosciences), CD11c APC(S-HCL-3, BD Biosciences), and CD14 APC (TüK4, Caltag) mAbs. Cell purity was >98%.

Coculture of DCs and naïve CD4⁺ T cells. Monocytes were isolated from PBMCs by negative selection using Monocyte Isolation Kit II (Miltenyi Biotec). DCs were generated by culturing monocytes with 50 ng/ml IL-4 (R&D) and 100 ng/ml GM-CSF (Leukine) in RPMI 1640 complete medium (GIBCO) containing 1% L-glutamine, 1% penicillin/streptomycin, 50 μM 2-mercaptoethanol, 1% sodium pyruvate, 1% nonessential amino acids (all from Sigma), 25 mM HEPES p7.2 and 10% heat-inactivated FBS (Hyclone) in 6 well plates (2×10⁶ cells/3 ml/well). Cytokines were added every 2 days. At day 6, dendritic cells were stimulated with irradiated CD40L-transfected L-cells, PGN (5 μg/ml, InvivoGen), LPS (50 ng/ml, Sigma-Aldrich), flagellin (20 ng/ml, InvivoGen), CL097 (imidazoquinoline compound. 5 μg/ml, InvivoGen), heat killed Escherichia coli (10⁸/ml, Invitrogen), heat killed Staphylococcus aureus (10⁸/ml—InvivoGen), or heat killed Porphyromonas gingivalis (10⁸/ml—InvivoGen). After 6 hours stimulation, DCs exposed to TLR-ligands or heat-killed bacteria were harvested and carefully washed: DCs stimulated with CD40L-transfected L cells were recovered as CD11c⁺CD40L⁻DCs by FACS after staining with CD11c APC(S-HCL-3, BD Biosciences) and CD40L PE (TROP1, BD Biosciences) mAbs. Activated DCs (1.3×10³ cells/well) were cultured for 7 d with allogeneic naïve CD4 T cells (4×10⁴ cells/well) in 96 well round bottom plates in RPMI complete medium. In some experiments, 10 μg/ml anti-IL-12p40 (C8.6, eBiosciences) or anti-IL-12p70 (M122, Pierce) blocking mAbs were added to the culture. For the assessment of IL-12 secretion, DCs harvested at 6 h stimulation were cultured for another 24 h in flat bottom 96 well plates (2×10⁵ cells/well), and the secreted IL-12 was measured with IL-12p70 ELISA kit (eBiosciences).

Stimulation of naive CD4 T cells via CD3/CD28. Naïve CD4 T cells (1×10⁵ cells/well) were stimulated with plate-bound CD3 mAb (5 μg/ml, OKT3, ATCC) and soluble CD28 mAb (1 μg/ml. CD28.2, BD Biosciences) in flat-bottomed 96 well plates in RPMI complete medium in the presence of human recombinant cytokines: IL-1β (R&D), IL-6 (R&D), IL-10 (R&D), IL-12 (R&D), IL-18 (R&D), IL-23 (eBiosciences), IL-27 (R&D), TNF (R&D), (10 ng/ml each or at indicated concentration), or IFN-α (IFN-α2b. 500 IU/ml, Schering). Activated CD4 T cells were analyzed at day 7. In some experiments, naïve CD4 T cells were labeled with 1 carboxyfluorescein diacetate succinimidyl ester (Molecular Probes) to track the proliferation of T cells.

Intracellular cytokine staining Naïve CD4 T cells stimulated for 7 d were restimulated with phorbol myristate acetate (25 ng/ml, Sigma-Aldrich) and ionomycin (1 μg/ml, Sigma-Aldrich) for 6 hours in the presence of GolgiPlug (BD Biosciences) for the last 4 hours. Cells were then fixed and permeabilized using Cytofix/Cytoperm Fixation/Permeabilization kit (BD Biosciences) and the expressed cytokines in the cytoplasm were analyzed with IL-21 PE or APC (3A3-N2, eBiosciences), IL-17A PE (64DEC17, eBiosciences), and IFNγ APC (B27, BD Biosciences) mAbs. Cells were acquired on FACS Calibur or FACS Cantoll after fixation with 1% paraformaldehyde. Cytokine expression in activated CD4⁺ T cells (FSC^{high} cells) was analyzed with FlowJo software (TreeStar).

IL-21 secretion from activated CD4⁺ T cells. Activated CD4⁺ T cells were sorted at day 7 as FSC^{high} cells (CD3/28 stimulation), or CD11c^{low}FSC^{high} cells (co-culture with DCs) after staining with CD11c APC(S-HCL-3, BD Biosciences) to remove the residual CD11c DCs. Sorted CD4 T cells were restimulated with plate-bound CD3 mAb (5 μg/ml) and soluble CD28 mAb (1 μg/ml) in 96 flat bottom well plates (5×10⁴ cells/well) in Yssel media (Gemini) supplemented with 10% FBS. After 24 hours, produced IL-21 levels were assessed using Luminex assay.

Development of human IL-21 bead-based assay using Luminex technology. PCR was used to insert a sequence bound by TGCTGGCTA and TGA and encoding mouse signaling lymphocytic activation molecule family member 1 signal peptide (gb|EDL39054.11) residues 1-24, GL, gb|ABN54273.1| cellulosome anchoring protein residues 1050-1219, LEAD, and gb|AAG29348.1| human interleukin 21 residues 30-162 into the Hind III—Not I interval of the mammalian expression vector pCDM8 (Seed, 1987). This vector directed secretion of a cohesin-IL-21 fusion protein. A vector directing the secretion of native human IL-21 was engineered by inserting ref|NM_{—}021803.1| residues 47-535 preceded by CACC into the Nhe I—Not I interval of pIRES2-DsRed2 (Clontech). Secreted proteins were produced using the FreeStyle™ 293 Expression System (Invitrogen) according to the manufacturer's protocol based on 1 mg total plasmid DNA with 1.3 ml 293 Fectin reagent/L of transfection. Transfected cells were cultured for 3 days, the culture supernatant was harvested and fresh 293 Freestyle™ media (Invitrogen) with 0.5% penicillin/streptomycin (Biosource) added with continued incubation for 2 days. The culture supernatant (1 L) was loaded onto a 20 ml Q Sepharose column (GE Healthcare) washed with PBS and then eluted with PBS+1M NaCl pH 7.4. The eluted fraction was passed over a custom built 1 ml anti-Cohesin mAb column, washed with PBS and eluted with 0.1 M glycine pH 2.7, and then dialyzed versus DPBS. The protein was analyzed by SDS-PAGE gel and concentration was based on theoretical extinction coefficient at 280 nm.

Mouse mAbs against human IL-21 were generated by a rapid, repetitive immunization strategy. Briefly, six-week-old BALB/c mice were immunized by foot pad injection, with 10 μg of cohesin-IL-21 fusion protein and Ribi or CpG (1017 ISS, Dynavax) adjuvant, 7-9 times over a course of 30-40 days. Upon observation of enhanced sera titers, a boost of 10 μg was given three to four days prior to harvesting inguinal and popliteal draining lymph nodes for PEG-induced somatic fusion with P3×63, Ag8.653 (ATCC CRL-1580) and/or SP2/O—Ag 14 myeloma cell lines, which had been adapted to lower serum tolerance. Hybridoma supernatants at a 1:25 dilution were screened by direct ELISA with plates coated at 0.5 μg/ml with a control cohesin fusion protein or at or 0.2 μg/ml cohesin-IL-21. Supernatants were also screened in a capture ELISA format using plates coated with goat anti-mouse IgG to anchor the monoclonal antibodies and detection with biotinylated IL-21 at 0.0625 μg/ml followed by Neutravidin-HRP. The hybridomas yielding the most potent antibodies were single cell cloned and scaled for production of pure antibody. The mAbs were tested in a checkerboard ELISA format to establish pairs that could detect IL-21. The mAbs were bound to plates at 2 μg/ml and then incubated with IL-21 at 2 ng/ml and 20 pg/ml, then with biotinylated mAbs at 100 ng/ml followed by detection with Neutravidin-HRP. mAbs that successfully paired were further screened in this ELISA by IL-21 titration from 100 ng/ml to 45 pg/ml and detection with 100 ng/ml of the biotinylated mAb partner. Those antibodies that were most sensitive in ELISA were conjugated to beads (Luminex Corporation protocol for two-step carbodiimide coupling of protein to carboxylated microspheres, January 2006) and incubated with a titration series of recombinant human IL-21 from 4000 pg/ml to 1 pg/ml, as well as dilutions of supernatant from CD4⁺ T cells stimulated with ionomycin and PMA, expected to contain native, human IL-21. The detecting mAbs were biotinylated and used at 0.5 μg/ml. The selected mAb pair detected IL-21 secreted from 293F cells transfected with the IL-21 expression vector. Cohesin-IL-21, which was significantly more potent than recombinant IL-21 from two commercial sources, was used as the standard.

The final Luminex assay was sensitive to 1 pg/ml hIL-21 over a range to at least 4000 pg/ml. The IL-21 pairs were tested on the Upstate Beadlyte Human 26 plex multiplex standards. There was no cross-reactivity with any of the analytes. The IL-21 pair could also be multiplexed with the Upstate human 22 plex. Luminex bead conjugations and general assay conditions are detailed by Giavedoni, et al. (Giavedoni, 2005). SeroMAP beads (region 26) were used with optimal coupling of 1×10⁷ beads at 5 μg mAb in 500 μl 50 mM MES pH 5.0. Biotinylation of detector mAb was done at 25×NHS-LC biotin (Pierce) and used at 0.5 μg/ml in the assay. Phycolink Strepavidin R-Phycoerythrin PJ31S from Prozyme, at 2 μg/ml, was used as the reporter.

Co-culture of T and B cells. Activated CD4⁺ T cells were sorted as described above, and co-cultured with autologous memory B cells (4×10⁴ cells/well each) in 96-well round-bottom plates in Yssel medium/10% FBS in the presence of endotoxin-reduced SEB (0.25 ng/ml; Toxin technology, Inc.). In some experiment anti-IL-2 mAb (PAB956, BIIR), anti-IL-4 mAb (MP4-25D2, BD), anti-IL-10 mAb (PAB548, BIIR), anti-IFN-γ mAb (B27, BD Biosciences), ICOS-L-mIgFc (Ancell), IgG1Fc or IL-21R/Fc (both from R&D systems) were added to the culture. Igs (IgM, IgG and IgA) produced in the cultures were analyzed by ELISA at day 6 or 14.

Ig ELISA. For measurement of Igs, culture supernatant were incubated for 2 h at room temperature in 96 well microtiter plates (Nunc) coated with 5 μg/ml goat anti-human IgM, IgG, IgA Abs (all from SouthernBiotech, Inc.). After washing, plates were incubated at room temperature for 1 h with alkaline phosphatase-conjugated goat anti-human IgM, IgG, or IgA Abs (at a final dilution of 1/2000, 1/2500, or 1/2500, respectively. all from SouthernBiotech, Inc.). Then, the plates were incubated with p-nitrophenyl phosphate (Sigma) after extensive wash. Reaction was stopped with NaOH 3N, and optical density was read by SpectraMax plate reader (Molecular Devices).

PBMC cultures with SEB. Purified fresh PBMCs (2.5×10⁵ cells/well) were cultured with SEB (0.1 μg/ml) in 96-well plates for 48 h in the presence or absence of anti-IL-12p40 or IL-12p70 mAbs, and the secreted cytokines were measured by Luminex.

Analysis of CD4⁺ T Cell Phenotype. To analyze CXCR5⁺ T cell subpopulations, fresh whole blood samples obtained from autoimmune disease patients and age-matched healthy subjects were stained with anti-CXCR5-Alexa 488, CCR6-PE, CD45RA-ECD, CXCR3-PC5, CCR4-PC7, ICOS-APC, CD3-AF700, CD8-APC H7, CD45RA Pacific Blue, and CD45 Pacific Orange mAbs. Cells were acquired with FACSAria, and analyzed with FlowJo software (TreeStar).

IL-12 as a key factor for the development of B-helper T cells. IL-21 is a T cell- and NKT cell-cytokine, which acts on many cells of the immune system. Particularly, IL-21 promotes the growth and the differentiation of B cells towards antibody-secreting plasma cells. FIG. 1 shows that naïve CD4+ T cells primed in the presence of IL-12 produce large amounts of IL-21. Briefly, naïve CD4+ T cells were stimulated for 7 days with plate-bound anti-CD3/CD28 mAbs in the presence of cytokines (10 ng/ml, except IFN-α at 500 IU/ml). IL-113, IL-6, IL-10, IL-18, IL-27, TNF-α, and IFN-α did not induce CD4+ T cells able to secrete IL-21. However, IL-12 promoted the development of CD4+ T cells able to produce IL-21. Upon re-activation through CD3/CD28, CD4+ T cells primed with IL-12 were able to secrete nanogram amounts of IL-21 (3.3±0.4 ng/ml in a culture of 5×10⁴ cells/200 μl. Mean±s.e.m. n=5), while those primed with IL-23 secreted only picogram amounts (40±4 pg/ml. Mean±s.e.m. n=5). Thus, IL-12 potently induces naïve CD4+ T cells to produce IL-21.

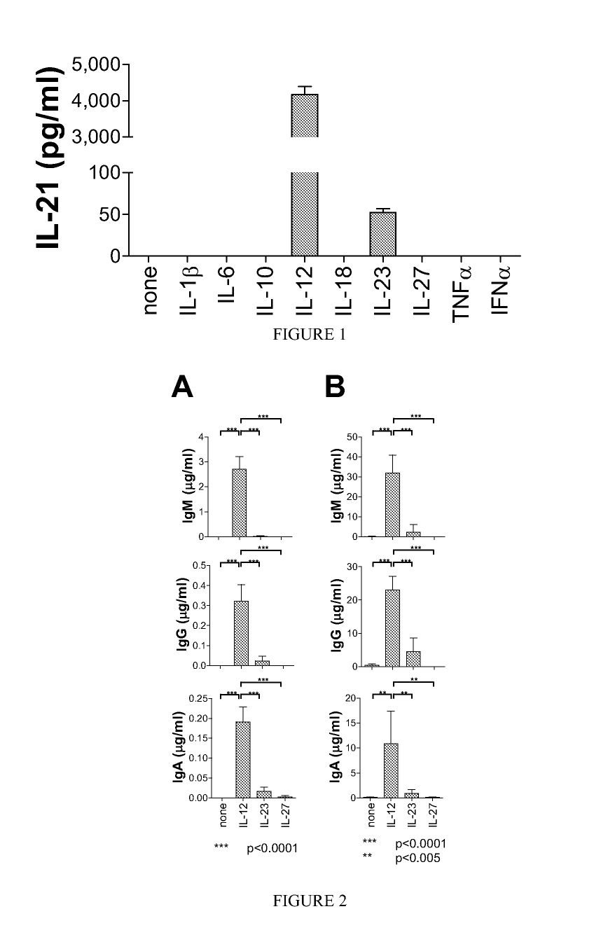

FIG. 2 shows that naïve CD4+ T cells primed in the presence of IL-12 are able to induce autologous B cells to produce immunoglobulins. Briefly, naïve CD4+ T cells activated by anti-CD3/CD28 mAbs in the presence of IL-12 or other cytokines were sorted at day 7, and cocultured with autologous blood IgD+CD27− naïve B cells pre-activated with anti-IgM mAb and CpG (TLR-9 ligand). Staphylococcal enterotoxin B (SEB), a superantigen, was added to induce T-B interactions, and the secreted Igs were measured at day 14. CD4+ T cells primed with no cytokine failed to induce naïve B cells to secrete Igs (A, none). In contrast, CD4+ T cells primed with IL-12 induced naïve B cells to secrete Igs, including IgM, IgG, and IgA. Naïve B cells co-cultured with CD4+ T cells primed with IL-23 produced significantly lower amounts of Igs.

Similarly, naïve CD4+ T cells primed with IL-12 induced Ig production from IgD-CD27+ memory B cells at significantly higher amounts than CD4+ T cells primed with IL-23 (B). Thus, IL-12 induces CD4+ T cells to become cells capable of helping B cells.

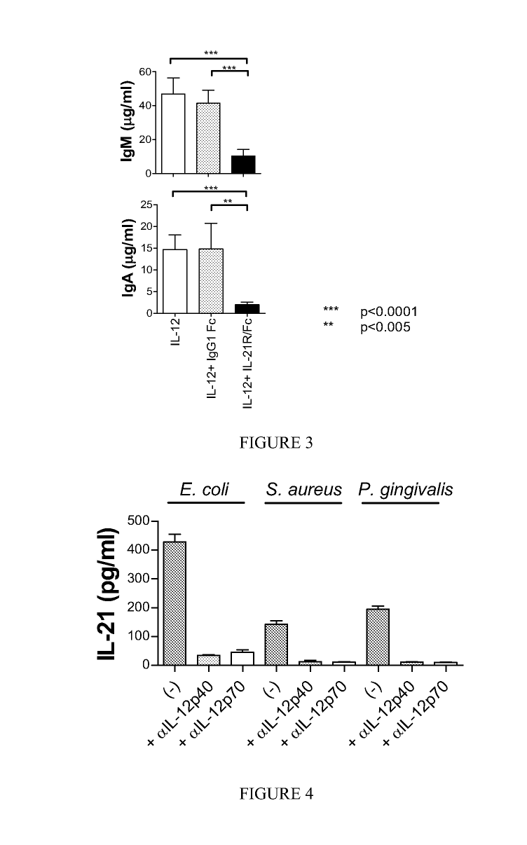

FIG. 3 shows that CD4+ T cells primed in the presence of IL-12 induce B cells to produce immunoglobulins in a manner dependent on IL-21. Briefly, soluble IL-21 receptor/Fc chimeric protein, which inhibits the function of IL-21, was added to the co-cultures of B cells and the CD4+ T cells primed with IL-12. Blocking IL-21 significantly inhibited B cells to secrete Igs. Thus, IL-21 secreted from CD4+ T cells primed with IL-12 play a fundamental role in the induction of antibody production by B cells.

FIG. 4 shows that activated DCs induce IL-21-producing CD4+ T cells through IL-12. Briefly, DCs were incubated for 6 h with heat-killed bacteria including E. coli (gram negative), S. aureus (gram positive), and P. gingivalis (gram positive), and then cultured with allogeneic naive CD4⁺ T cells. Activated CD4⁺ T cells sorted at day 7 were re-stimulated with anti-CD3/CD28 mAbs for 24 h to measure IL-21 secretion. CD4⁺ T cells primed with bacteria-activated DCs secreted more IL-21 than those primed with unstimulated DCs. Addition of anti-IL-12p40 blocking mAb, which inhibits both IL-12 and IL-23, during DC-T cell co-cultures significantly inhibited the development of IL-21-producing CD4⁺ T cells by bacteria-activated DCs. Notably, addition of anti-IL-12p70 blocking mAb, which inhibits IL-12 alone, was sufficient to significantly inhibit IL-21 secretion by CD4⁺ T cells (85±5% inhibition. Mean±s.e.m. n=5). Thus, the induction of IL-21-producing CD4⁺ T cells by DCs that sensed bacteria was mediated by IL-12.

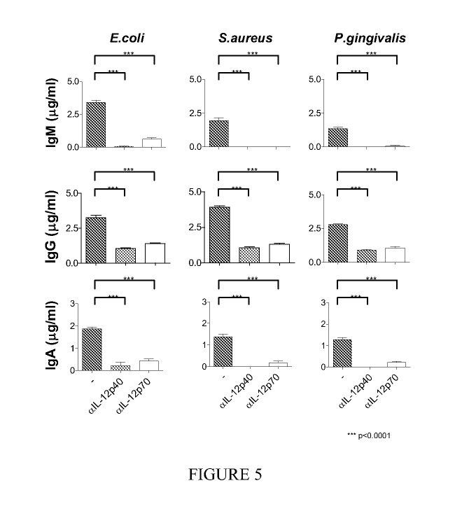

FIG. 5 shows that blocking IL-12 inhibits the development of T cells capable of helping B cells. Briefly, CD4+ T cells activated by culturing with bacteria-activated DCs were sorted at day 7 and co-cultured with autologous memory B cells. CD4+ T cells primed with bacteria-activated DCs induced B cells to produce Igs. The induction of Ig secretion by B cells was significantly impaired when anti-IL-12p40 mAb was added during DC-T cell co-cultures. However, blocking IL-12 with anti-IL-12p70 mAb was sufficient to inhibit the development of B-helper CD4+ T cells. Thus, IL-12 secreted by activated DCs is critical for the differentiation of naïve CD4+ T cells into B-helper T cells.

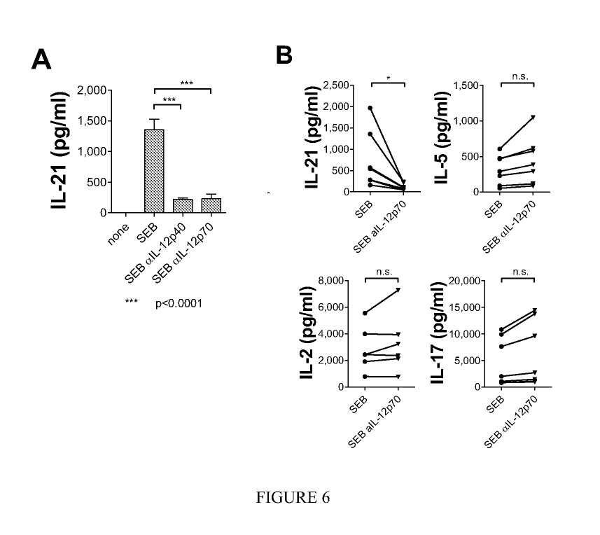

IL-12 controls IL-21 secretion. FIG. 6 shows that IL-12 controls the secretion of IL-21 secretion by memory CD4+ T cells. Briefly, mononuclear cells (PBMCs) were isolated from blood samples of healthy adults, and stimulated with staphylococcal enterotoxin B (SEB). Produced IL-21 levels were analyzed at 48 h of culture. PBMCs produced high amounts of IL-21 when cultured with SEB for 48 h (A. 740±250 pg/ml. Mean±s.e.m. n=7). Blocking IL-12 during the 48 h activation period with anti-IL-12p70 mAb resulted in a significant decrease of IL-21 secretion (A&B. 110±30 pg/ml. n=7. p<0.05). Blocking of both IL-12 and IL-23 with anti-IL-12p40 mAb decreased IL-21 secretion (120±40 pg/ml. n=6) at levels comparable to blocking IL-12 alone. Blocking IL-12 did not alter the secretion of other cytokines including IL-2, IL-5, and IL-17 (B). Thus, blocking IL-12 specifically inhibited the secretion of both IL-21 and IFN-γ. Thus, IL-12 directly acts on IL-21-producing memory CD4+ T cells and promotes the secretion of IL-21.

Increased frequency of functional B-helper T cells in the blood of autoimmune diseases. Example of autoantibody-associated autoimmune disease: Dermatomyositis (DM).

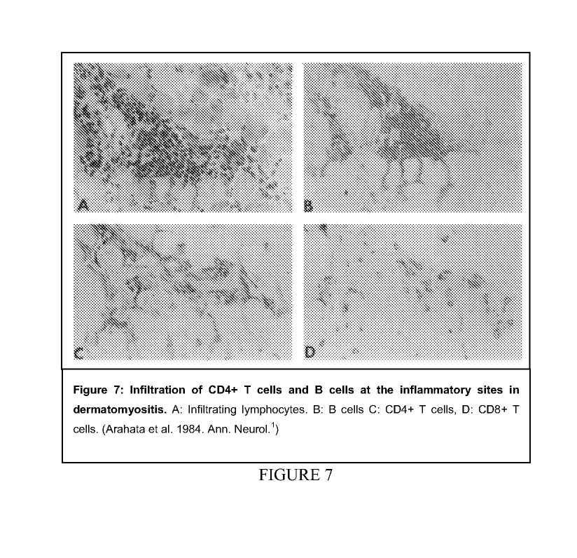

DM is an autoimmune inflammatory myopathy. Patients with DM display proximal muscle weakness and systemic inflammatory features including a characteristic skin rash. The incidence of JDM in the USA is 3.2 per million children per year. The average age at onset is 7 years, but 25% of patients are younger than 4 years at onset. In the USA, the ratio of girls to boys is 2.3 to 1. Adult DM is most common in the 40 to 60 year age range, and observed in about 2 out of 100,000 people in the US. CD4⁺ T cells and B cells predominantly accumulate at inflammatory sites in DM (FIG. 7). Many DM patients display a broad repertoire of autoantibodies, thus B cells have been proposed to be critical in DM pathogenesis. Actually, two recent pilot trials with Rituximab (anti-CD20 mAb) showed that deletion of B cells was beneficial to DM patients.

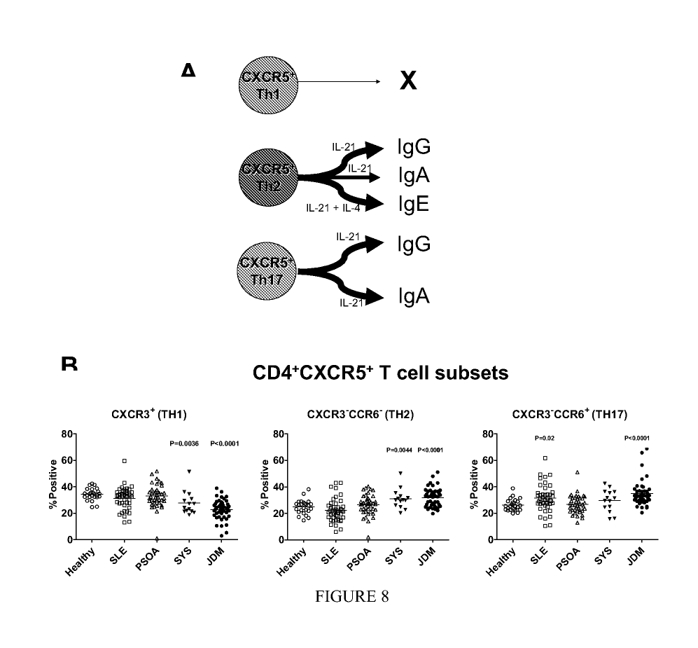

FIG. 8 demonstrates that autoimmune disease patients, including juvenile dermatomyositis (JDM) patients, display skewed CXCR5+ T cell subsets when compared to healthy controls.

Blood CD4+ T cells expressing CXCR5, a chemokine receptor, represent a CD4+ T cell subset specialized for antibody responses. CXCR5+CD4+ T cells secrete large amounts of IL-21 when stimulated with anti-CD3 mAb and ICOS. Within CXCR5+CD4+ T cells, three major subpopulations were identified: Th1 (CXCR3+), Th2 (CXCR3−CCR6−) and Th17 (CCR6+) cells. While Th2 and Th17 cells are efficient B cell helpers, Th1 cells are totally unable to help B cells (A).

Phenotypical analysis of PBMCs revealed that systemic arthritis (SYS) and JDM patients show less CXCR5+Th1 (CXCR3+) and more CXCR5+Th2 (CXCR3−CCR6−) cells than age-matched healthy controls (Shown in B). The frequencies of CXCR5+Th17 (CXCR3−CCR6+) cells were higher in JDM and SLE (systemic lupus erythematosus) than healthy controls. Furthermore, the frequencies of CXCR5+Th2 and Th17 cell populations were higher in JDM than those in other autoimmune diseases. CXCR5+Th2 cell population was significantly higher than in SLE, and the CXCR5+Th17 cell population was higher than in PSOA (Psoriatic arthritis). Remarkably, three of four JDM patients who displayed the highest CXCR5+Th17 cells were among the sickest and most refractory patients in the studied cohort (persistent elevation of muscle enzymes, CMAS<40 and persistent skin rashes). The frequency of CXCR5+Th1 cells in JDM was significantly lower than in the other two autoimmune diseases. Thus, CXCR5+CD4+ T cell subsets are skewed in SYS, SLE, and JDM towards Th2 and/or Th17 cells, which represent the most efficient B cell helpers. This immuno-dysregulation might contribute to the generation of pathogenic autoreactive B cells.

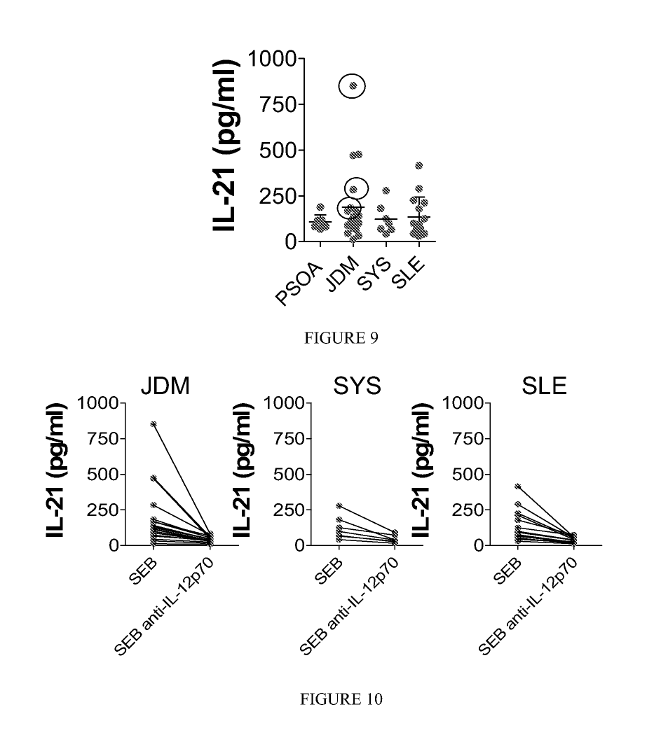

Increased IL-21 secretion by memory CD4+ T cells obtained from active JDM patients. FIG. 9 demonstrates that peripheral blood mononuclear cells obtained from active JDM patients secrete large amounts of IL-21. Briefly, fresh PBMCs were obtained from psoriatic arthritis (PSOA), JDM, SYS, and SLE pediatric patients, and stimulated with SEB. The produced IL-21 levels were measured at 48 h. Remarkably, three of five JDM patients who displayed the highest IL-21 secretion (indicated in circles) were among the active patients who required administration of predonisolone. Thus, blood cells of active JDM patients secrete higher amounts of IL-21 upon activation.

FIG. 10 shows that IL-12 is the key cytokine also in the induction of IL-21 secretion by PBMCs obtained from autoimmune disease patients. Briefly, PBMCs from JDM, SYS, and SLE patients were stimulated with SEB in the presence or absence of IL-12-neutralizing mAb, and the secreted IL-21 levels were analyzed. IL-21 secretion was significantly inhibited by blocking IL-12 in the cultures of PBMCs from all the tested autoimmune diseases (JDM: n=20, p=0.0027; SYS: n=7, p=0.015; SLE: n=15, p=0.002. Paired t-test), which is consistent with the finding with samples obtained from healthy adults. Thus, IL-12 plays an essential role in the induction of IL-21 secretion from memory CD4+ T cells in autoimmune diseases including JDM, SYS, and SLE.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for treating an autoimmune disorder comprising administering to a subject having an autoimmune disorder, an effective amount of a therapeutic composition comprising an IL-12 inhibitor in an amount sufficient to block B-Helper T cell activity.

2. The method of claim 1, wherein the IL-12 inhibitor comprises at least one blocking anti-IL-12 antibody or fragment thereof.

3. The method of claim 1, further comprising a pharmaceutically acceptable carrier.

4. The method of claim 1, wherein the IL-12 inhibitor comprises an IL-12 inhibitory antibody administered in a dosage of from 1 to 1,000 mg per dose.

5. The method of claim 1, wherein the IL-12 inhibitor comprises an IL-12 inhibitory antibody and the subject receives the antibody in repeated dosages.

6. The method of claim 1, wherein the IL-12 inhibitor comprises an anti-IL-12 antibody selected from the group consisting of subhuman primate antibody, murine monoclonal antibody, chimeric antibody, humanized antibody, and human antibody.

7. The method of claim 1, wherein the IL-12 inhibitor comprises an RNAi, siRNA or other nucleic acid inhibitor of IL-12.

8. The method of claim 1, wherein the autoimmune disease is selected from the group consisting of acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis.

9. The method of claim 1, further comprising separately administering a secondary therapeutic directed against T-cells, B-cells, plasma cells, or macrophages or inflammatory cytokines.

10. A method of enhancing an antigen-specific B cell response against an antigen comprising:
isolating naïve CD4⁺ T cells; and
maturing the naïve CD4⁺ T cells in the presence of antigen-loaded dendritic cells with an effective amount of IL-12 sufficient to develop and activate B-helper T cells, wherein the IL-12 treated B-Helper T cells secrete nanomolar amounts of IL-21.

11. The method of claim 10, wherein the naïve CD4⁺ T cells are isolated from peripheral blood mononuclear cells by negative selection.

12. The method of claim 10, wherein the naïve CD4⁺ T cells are obtained from peripheral blood mononuclear cells by negative selection using antibodies against CD8 and one or more antibodies against CD11b, CD11c, CD14, CD15, CD16, CD19, CD45RO, CD56 and HLA-DR.

13. The method of claim 10, wherein the antigen is selected from a virus, a bacteria, a fungi, a cancer or a toxin.

14. A method of modulating autoimmune diseases comprising:
identifying a patient suspected of needing therapy for an autoimmune disorder caused by the secretion of autoimmune antibodies by B cells; and
treating the patient with an amount of an anti-IL-12 inhibitor sufficient to inhibit CD4⁺ B helper T cells.

15. The method of claim 14, wherein the anti-IL-12 inhibitor comprises an anti-IL-12p40 mAb, an anti-IL-12p70 mAb, anti-IL-12 receptors, soluble inactive IL-12 and combinations thereof.

16. The method of claim 14, wherein the anti-IL-12 inhibitor comprises an IL-12 receptor antagonist.

17. The method of claim 14, wherein the CD4⁺ B helper T cells are selected by negative selection.

18. The method of claim 14, wherein the CD4⁺ B helper T cells are obtained from peripheral blood mononuclear cells by negative selection using antibodies against CD8 and one or more antibodies against CD11b, CD11c, CD14, CD15, CD16, CD19, CD45RO, CD56 and HLA-DR.

19. The method of claim 14, wherein the CD4⁺ B helper T cells are activated in the presence of activated dendritic cells.

20. The method of claim 14, wherein the autoimmune disease is selected from systemic lupus erythematosus, dermatomyositis, juvenile dermatomyositis, arthritis, systemic arthritis and psoriatic arthritis.

21. The method of claim 14, wherein the IL-12 inhibitor is provided to a subject suspected of susceptibility of an autoimmune disease prior to the development of autoimmune antibodies.

22. A B cell Helper T cell made by the method comprising:
isolating naïve CD4⁺ T cells; and
maturing the naïve CD4 T cells in the presence of activated dendritic cells expressing a target antigen in the presence of IL-12, wherein the matured CD4⁺ B-helper T cells release nanomolar amounts of IL-21 in response to antigen.

23. A method of regulating B cell proliferation, maturation and activation into immunoglobulin secreting cells by exposing IL-21 secreting B-Helper T cells to a composition comprising an IL-12 inhibitor.

24. The method of claim 23, wherein the IL-12 inhibitor reduces the secretion of both IL-21 and IFN-γ by CD4⁺ B-Helper T cells.

25. A method of enriching B Helper T cells comprising incubating naïve CD4 T cells in the with an amount of IL-12 sufficient to trigger the release of nanomolar amounts of IL-21.
